(19) United States
(12) Patent Application Publication
(10) Pub. No.: US 2026/0183139 A1
(43) Pub. Date: Jul. 2, 2026

(54) MULTIFUNCTIONAL CONDOM WITH ANTIBACTERIAL, STERILIZATION, AND VAGINAL MUSCLE TIGHTENING PROPERTIES

(71) Applicants: Meesue Kim, Woodinville, WA (US); Yoo Seung Kim, Bangkok (TH); Miss Kawinyarat Mardpiratchata, Bangkok (TH)

(72) Inventors: Meesue Kim, Woodinville, WA (US); Yoo Seung Kim, Bangkok (TH); Miss Kawinyarat Mardpiratchata, Bangkok (TH)

(73) Assignee: Meesue Kim, Woodinville, WA (US)

(21) Appl. No.: 18/473,272

(22) PCT Filed: Sep. 24, 2023
(86) PCT No.: PCT/US2023/074981

§ 371 Date: Sep. 24, 2023

(51) Int. Cl. A61F 6/04

(52) U.S. Cl. CPC A61F 6/04

(57) ABSTRACT

The present invention relates generally to condoms and, more specifically, to a condom coated with a composition that provides multiple benefits including lubrication, sterilization, antibacterial properties, and vaginal muscle tightening.

### FIELDS

The present invention relates to the field of sexual health products, specifically to condoms designed to provide additional benefits such as lubrication, sterilization, antibacterial properties, and vaginal muscle tightening

### BACKGROUND OF THE INVENTION

Condoms are widely used as a method of contraception and to prevent the transmission of sexually transmitted infections (STIs). Most condoms are lubricated to reduce friction and prevent breakage during use. However, the lubricants used on condoms do not typically provide any additional benefits. There is a need for a condom that provides additional benefits such as sterilization, antibacterial properties, and vaginal muscle tightening.

### BRIEF SUMMARY OF THE INVENTION

The present invention provides a condom coated with a composition comprising a mixture of ingredients that provide multiple benefits including lubrication, sterilization, antibacterial properties, and vaginal muscle tightening.

### DETAILED DESCRIPTION OF THE INVENTION

The present invention encompasses a condom coated with a composition designed to offer a range of functionalities: lubrication, sterilization, antibacterial properties, and vaginal muscle tightening. The condom composition disclosed herein is provided as a non-limiting example of one embodiment of the invention that effectively realizes these functionalities using a specific mixture of ingredients and the ingredients can be grouped into several major functionalities:
1. LUBRICATION AND MOISTURIZATION:
   Propanediol
   Butylene Glycol
   Hydrolyzed Sodium Hyaluronate
   Allantoin
2. Sterilization and Antibacterial:
   Dendropanax Morbiferus Leaf Extract
   Astragalus Membranaceus Root Extract
   Melia Azadirachta Leaf Extract
   Phyllanthus Emblica Extract
   Boswellia Serrata Extract
3. Vaginal Muscle Tightening:
   Trifolium Pratense (Clover) Extract
4. Texture and Consistency:
   Acrylates/C10-30 Alkyl Acrylate Crosspolymer
   Hydroxyethyl Acrylate/Sodium Acryloyldimethyl Taurate Copolymer
   Xanthan Gum
5. pH Adjustment:
   Tromethamine
6. Preservative Booster:
   1,2-Hexanediol
   Disodium EDTA
7. Anti-inflammatory and Skin Soothing:
   Niacinamide
   Allantoin
   Adenosine
   Lactobionic Acid
8. Skin Health and Anti-aging:
   Garcinia Cambogia Fruit Extract
   Phyllanthus Emblica Extract
   Adenosine
9. Emulsification:
   C12-14 PARETH-12

While the example composition provided below includes specific ingredients found to be effective in realizing the desired functionalities, it is understood that the invention is not limited to these specific ingredients or their proportions. Other ingredients with similar properties may be substituted, and the proportions of the ingredients may be adjusted, to achieve the same or similar functionalities without departing from the scope of the invention. I provide list of applicable ingredients together with composition comprises a mixture of the ingredients.:
Dendropanax Morbiferus Leaf Extract: It has antibacterial, anti-inflammatory, and antioxidant properties. This extract helps in fighting bacterial infections and also helps in reducing inflammation.
Propanediol: It is a natural, skin-friendly, preservative-boosting humectant that is used as a solvent for the other ingredients. It also provides lubrication to the condom.
1,2-Hexanediol: It is a humectant and coupling agent that helps in keeping the product from drying out and also acts as a preservative.
Niacinamide: It is a form of vitamin B3 that has anti-inflammatory and antioxidant properties. It helps in reducing inflammation and oxidative stress.
Acrylates/C10-30 Alkyl Acrylate Cross-polymer: It is a gelling agent that helps in thickening the formulation and provides a smooth texture to the condom.
Tromethamine: It is a buffering agent that helps in maintaining the pH of the formulation.
C12-14 PARETH-12: It is an emulsifying agent that helps in mixing the oil and water components of the formulation.
Allantoin: It is a skin-soothing agent that has anti-inflammatory properties. It helps in reducing irritation and inflammation of the skin.
Butylene Glycol: It is a humectant and solvent that helps in keeping the product from drying out and also helps in the penetration of the other ingredients into the skin.
Adenosine: It is a yeast-derived ingredient that has anti-inflammatory and skin-restoring properties. It helps in reducing inflammation and promoting the healing of the skin.
Disodium EDTA: It is a chelating agent that helps in preventing the breakdown of the other ingredients in the formulation.
Hydrolyzed Sodium Hyaluronate: It is a form of hyaluronic acid that has moisturizing and skin-conditioning properties. It helps in maintaining the hydration of the skin and also provides lubrication to the condom.
Hydroxyethyl Acrylate/Sodium Acryloyldimethyl Taurate Copolymer: It is a gelling agent that helps in thickening the formulation and provides a smooth texture to the condom.
Lactobionic Acid: It is a polyhydroxy acid that has antioxidant and humectant properties. It helps in reducing oxidative stress and keeping the product from drying out.
Xanthan Gum: It is a thickening agent that helps in providing a smooth texture to the condom.
Astragalus Membranaceus Root Extract: It is a traditional Chinese medicine that has anti-inflammatory, antioxidant, and immune-boosting properties. It helps in reducing inflammation, oxidative stress, and enhancing the immune response.
Boswellia Serrata Extract: It is an extract obtained from the resin of the Boswellia serrata tree that has anti-inflammatory and analgesic properties. It helps in reducing inflammation and pain.
Garcinia Cambogia Fruit Extract: It is an extract obtained from the fruit of the Garcinia cambogia tree that has anti-inflammatory and antioxidant properties. It helps in reducing inflammation and oxidative stress.
Melia Azadirachta Leaf Extract: It is an extract obtained from the leaves of the Melia azadirachta tree that has antibacterial, anti-inflammatory, and antioxidant properties. It helps in fighting bacterial infections and reducing inflammation and oxidative stress.
Phyllanthus Emblica Extract: It is an extract obtained from the fruit of the Phyllanthus emblica tree that has antibacterial, anti-inflammatory, antioxidant, and vaginal muscle tightening properties. It helps in fighting bacterial infections, reducing inflammation and oxidative stress, and tightening the vaginal muscles.
Trifolium Pratense (Clover) Extract: It is an extract obtained from the flowers of the Trifolium pratense plant that has estrogen-like effects. It helps in tightening the vaginal muscles.

The composition may be prepared by mixing the above-mentioned ingredients in the following proportions:
Dendropanax Morbiferus Leaf Extract: 5-10%
Propanediol: 10-20%
1,2-Hexanediol: 3-5%
Niacinamide: 2-5%
Acrylates/C 10-30 Alkyl Acrylate Crosspolymer: 2-5%
Tromethamine: 1-2%
C12-14 PARETH-12: 1-3%
Allantoin: 0.5-2%
Butylene Glycol: 3-10%
Adenosine: 0.1-1%
Disodium EDTA: 0.1-1%
Hydrolyzed Sodium Hyaluronate: 2-5%
Hydroxyethyl Acrylate/Sodium Acryloyldimethyl Taurate Copolymer: 1-5%
Lactobionic Acid: 1-5%
Xanthan Gum: 0.1-1%
Astragalus Membranaceus Root Extract: 1-5%
Boswellia Serrata Extract: 1-5%
Garcinia Cambogia Fruit Extract: 1-5%
Melia Azadirachta Leaf Extract: 1-5%
Phyllanthus Emblica Extract: 1-5%
Trifolium Pratense (Clover) Extract: 1-5%

The composition is mixed and applied to the surface of the condom during manufacturing procedure.

An example composition of the invention which was made by mixing the following ingredients in the given proportions has been tested:
Dendropanax Morbiferus Leaf Extract: 8%
Propanediol: 15%
1,2-Hexanediol: 4%
Niacinamide: 3%
Acrylates/C10-30 Alkyl Acrylate Crosspolymer: 3%
Tromethamine: 1.5%
C12-14 PARETH-12: 2%
Allantoin: 1%
Butylene Glycol: 7%
Adenosine: 0.5%
Disodium EDTA: 0.5%
Hydrolyzed Sodium Hyaluronate: 3%
Hydroxyethyl Acrylate/Sodium Acryloyldimethyl Taurate Copolymer: 3%
Lactobionic Acid: 3%
Xanthan Gum: 0.5%
Astragalus Membranaceus Root Extract: 3%
Boswellia Serrata Extract: 3%
Garcinia Cambogia Fruit Extract: 3%
Melia Azadirachta Leaf Extract: 3%
Phyllanthus Emblica Extract: 3%
Trifolium Pratense (Clover) Extract: 3%

The ingredients are mixed well and applied to the surface of the condom.

The following is the method of making the product:
1. Provide a condom.
2. Prepare the coating composition by mixing the ingredients mentioned in the detailed description.
3. Apply the coating composition to the inner and/or outer surface of the condom. The composition can be applied in a liquid, gel, or cream form.

1. A condom comprising a coating composition that provides lubrication, sterilization, antibacterial properties, and vaginal muscle tightening.

2. The condom of claim 1, wherein the coating composition is in the form of a dry powder.

3. The condom of claim 1, wherein the coating composition is in the form of a wet gel or cream.

4. The condom of claim 1, wherein the coating composition is applied to the inner surface of the condom.

5. The condom of claim 1, wherein the coating composition is applied to the outer surface of the condom.

6. The condom of claim 1, wherein the coating composition comprises one or more ingredients selected from the group consisting of [list of ingredients].

7. A method of making a condom, comprising the steps of providing a condom and applying a coating composition to the inner and/or outer surface of the condom, wherein the coating composition provides lubrication, sterilization, antibacterial properties, and vaginal muscle tightening.

8. The condom of claim 1, wherein the composition comprises one or more ingredients selected from the group consisting of: propanediol, butylene glycol, hydrolyzed sodium hyaluronate, allantoin, dendropanax morbiferus leaf extract, astragalus membranaceus root extract, melia azadirachta leaf extract, phyllanthus emblica extract, boswellia serrata extract, trifolium pratense (clover) extract, acrylates/C10-30 alkyl acrylate crosspolymer, hydroxyethyl acrylate/sodium acryloyldimethyl taurate copolymer, xanthan gum, tromethamine, 1,2-hexanediol, disodium EDTA, niacinamide, lactobionic acid, garcinia cambogia fruit extract, adenosine, C12-14 PARETH-12, and combinations thereof.

9. The condom of claims 1 or 8, wherein the composition provides antioxidant properties.

10. The condom of claims 1 or 8, wherein the composition provides anti-inflammatory properties.

11. A method of manufacturing a condom, comprising the steps of providing a condom and applying a coating composition to the inner and/or outer surface of the condom, wherein the coating composition provides lubrication, sterilization, antibacterial properties, and vaginal muscle tightening.

12. The method of claim 11, wherein the coating composition is applied in a powder, liquid, gel, or cream form.

13. The condom of any one of claims 1 to 11, wherein the coating composition comprises one or more ingredients with antibacterial properties.

14. The condom of any one of claims 1 to 11, wherein the coating composition comprises one or more ingredients with vaginal muscle tightening properties.

15. The condom of any one of claims 1 to 11, wherein the coating composition comprises one or more ingredients with soothing properties.
